(19) United States
(12) Patent Application Publication
(10) Pub. No.: US 2026/0325007 A1
(43) Pub. Date: Oct. 22, 2026

(54) DEVICE, METHOD AND SYSTEM FOR TRACKING EXTREMITY IMPAIRMENT

(71) Applicant: McMaster University, Hamilton (CA)

(72) Inventors: Jenna SMITH-TURCHYN, Anacaster, CA (US); Ethan Shen, Mississauga, CA (US); Stavros Gentile, Mississauga, CA (US); Mohamed Kasim, Mississauga, CA (US); Neema Sadeghpour, Toronto, CA (US)

(21) Appl. No.: 19/649,777

(22) Filed: Apr. 16, 2026

Related U.S. Application Data
(60) Provisional application No. 63/789,831, filed on Apr. 16, 2025.

(51) Int. Cl. A61B 5/107; A61B 5/00

(52) U.S. Cl. CPC A61B 5/1075; A61B 5/1072; A61B 5/1073; A61B 5/6887; A61B 5/7278

(57) ABSTRACT

A diagnostic medical device and a method for measuring anatomical features of a patient appendage are provided. The device comprises a support frame defining a passage therein, and a plurality of spaced-apart sensors carried by the support frame. The passage is configured to accommodate relative movement of a patient appendage along the passage. The plurality of sensors is directed inwardly into the passage and arranged to transversely surround the patient appendage when received in the passage. The sensors are configured to cooperate to obtain data representing a volume of a measured portion of the patient appendage by measuring a plurality of circumferences of the patient appendage at a respective plurality of longitudinal positions of the patient appendage relative to the passage when the patient appendage is received in the passage.

### CROSS-REFERENCE TO RELATED APPLICATION

This application claims priority to, and the benefit of, U.S. Provisional Patent Application No. 63/789,830 filed on Apr. 16, 2025, the teachings of which are hereby incorporated by reference.

### TECHNICAL FIELD

The present disclosure relates to biometric measurement devices and methods, and in particular, to a diagnostic medical device, method, and system for determining an appendage impairment.

### BACKGROUND

In 2022 approximately 28,600 Canadians were diagnosed with breast cancer (BRCA) [1]. Survivors of cancer, and in particular breast cancer, live with many physical and psychosocial treatment-related side effects for years after treatment [2, 3]. Upper extremity (UE) impairment is a leading cause of decreased function in BRCA survivors [4, 5]. Lymphedema affects approximately 40% of survivors and impaired shoulder range of motion has a prevalence of up to 67% [6, 7]. Risk factors for lymphedema development and UE range of motion impairment include breast surgery, lymph node dissection, radiotherapy, obesity, seroma formation following surgery, and undergoing chemotherapy [8, 9]. The large majority of BRCA survivors possess two or more of these risk factors.

While risk of lymphedema and UE range of motion limitations is high, these conditions are preventable, even reversible, if identified and treated early [5]. The current model of healthcare in Canada relies on the identification of significant arm swelling or UE range of motion impairments by a health professional to diagnose these impairments. However, systematic screening is lacking. This delays diagnosis, compounds impairment, and complicates management [10, 11]. Failure to recognize and properly manage these impairments can lead to significant and sustained functional limitations. This further leads to both decreased quality of life and participation in meaningful activities at home and at work [3-5, 7].

Self-care and self-management strategies empower individuals to actively manage their own health issues: preventing the development of secondary and tertiary disability, enabling better self-monitoring capability, and promoting adherence to intervention regimes [12-14]. Self-care strategies may also reduce healthcare costs [12-14].

Barriers to systematic and timely monitoring of UE impairment by health professionals include a lack of patient access to health professionals with knowledge of UE function, clinical time and resources, and patient time due to competing life demands [15]. Additionally, the volume of survivors makes it difficult for the current system to regularly assess and manage UE function. This is especially true as the incidence of these impairments increases with time post active treatment (e.g., lymphedema incidence increases from 13.5% at 2 years to 41.1% at 10 years post treatment) [6]. During this period survivors see their oncology team less frequently, if at all. Providing self-care strategies is vital as the incidence and burden of cancer is projected to increase substantially over the next decade, so providing self-care strategies for cancer survivors would help to limit and treat impairments [16].

Self-measurement of arm circumference using a tape measure by women with and without lymphedema has been found reliable for treating and managing the impairment [17, 18]. However, by manually measuring and recording arm circumference there is potential for random error during measuring and when recording scores over time [17, 18]. This process has also required users to interpret measurements over time and decide on appropriate next steps if changes occur. This can lead to further error as the minimum clinically important difference (MCID) for circumferential measurements is small, at 2 cm [18]. Incorrect interpretation can lead to missed or delayed detection of impairment onset, and more severe long-term limitations. While UE volume measurement is beneficial for lymphedema assessment and management, this is currently not done clinically due to the invasiveness of measurement techniques (submerging the entire UE in water) and there is no method to do volume measurement at home independently. Cancer survivors demonstrate need, acceptability and adherence to rehabilitation programs using technology [19]. However, no system exists to manage UE function over the short or long term.

While tape measure applications exist [20, 21] their reliability and validity are not well explored, and current ones do not measure properties that allow for the identification of lymphedema. There are goniometry [16, 22] applications to measure UE range of motion that demonstrate high inter and intra-rater reliability (ICC=0.995−1.0) [23, 24] and concurrent validity (ICC=0.998−0.999) [24-26]. However, these applications are not breast cancer specific, do not exist on a platform together, and do not tailor evidence-based recommendations in response to the measurements obtained over time. Therefore, current systems do not provide accurate and usable information to identify and address UE impairment in this population. Accessible, accurate, and easy to use self-care tools using technology are needed for survivors to reduce complications, track UE function, and identify and deal with changes early if they do occur to prevent functional limitation.

The background herein is included solely to explain the context of the disclosure. This is not to be taken as an admission that any of the material referred to was published, known, or part of the common general knowledge as of the priority date.

### SUMMARY

In accordance with an aspect of the disclosure, a diagnostic medical device is provided. The device comprises a support frame defining a passage therein, and a plurality of spaced-apart sensors carried by the support frame. The passage is configured to accommodate relative movement of a patient appendage along the passage. The plurality of sensors are directed inwardly into the passage and arranged to transversely surround the patient appendage when received in the passage. The sensors are configured to cooperate to obtain data representing a volume of a measured portion of the patient appendage by measuring a plurality of circumferences of the patient appendage at a respective plurality of longitudinal positions of the patient appendage relative to the passage when the patient appendage is received in the passage.

In an embodiment, the device further comprises an encoder carried by the support frame and configured to measure longitudinal movement of the patient appendage.

In an embodiment, the encoder comprises a roller for contacting and rolling along a surface of the patient appendage.

In an embodiment, the device further comprises at least one processing unit coupled to the plurality of sensors and configured to receive signals from the plurality of sensors, and to process the signals to generate the circumference of the patient appendage at each of the plurality of longitudinal positions.

In an embodiment, the at least one processing unit is further configured to calculate the volume of the measured portion of the patient appendage using the circumferences of the patient appendage at each of the longitudinal positions.

In an embodiment, the plurality of sensors is coupled to a communication port adapted for communication to a computing device.

In an embodiment, the plurality of sensors is coupled to the communications port through at least one memory adapted to store signals from the sensors for communication to the computing device.

In an embodiment, the plurality of sensors and the encoder are coupled to a communications port adapted for communication to a computing device.

In an embodiment, the plurality of sensors and the encoder are coupled to the communications port through at least one memory adapted to store signals from the sensors for communication to the computing device.

In an embodiment, the support frame comprises a ring.

In an embodiment, the support frame comprises a tube having an open first end and a closed second end.

In an embodiment, the support frame comprises a tube having an open first end and an open second end.

In an embodiment, the patient appendage is an arm, and a first longitudinal position of the plurality of longitudinal positions is at a wrist crease or an upper shoulder of the arm.

In an embodiment, the plurality of sensors are distance sensors.

In an embodiment, the plurality of sensors are Time of Flight sensors.

In another aspect of the disclosure, there is provided a method for measuring anatomical features of a patient appendage. The method comprises receiving, at a data processing system, sensor data representing a series of circumference measurements at a respective plurality of longitudinal positions along the patient appendage, and the data processing system using the circumference measurements to calculate a volume of a portion of the patient appendage between a first one of the longitudinal positions and a last one of the longitudinal positions.

In an embodiment, the method further comprises receiving, at the data processing system, encoder data representing the respective plurality of longitudinal positions along the patient appendage. The data processing system uses the encoder data in conjunction with the circumference measurements to calculate the volume of the portion of the patient appendage between the first one of the longitudinal positions and the last one of the longitudinal positions.

In an embodiment, the sensor data comprises distance measurements from a plurality of distance sensors circumferentially surrounding the patient appendage.

In an embodiment, the data processing system calculates the circumference measurements from the sensor data.

In an embodiment, the sensor data comprises precalculated circumference measurements.

In a further aspect of the disclosure, there is provided a computer readable memory storing computer executable instructions thereon that when executed by a computer cause the computer to perform the methods described herein.

In another aspect of the disclosure, there is provided a data processing system comprising at least one processor and memory coupled to the at least one processor. The memory contains instructions which, when executed by the at least one processor, cause the data processing system to implement the methods described herein.

In yet a further aspect of the disclosure, there is provided at least one tangible, non-transitory computer-readable medium embodying instructions which, when executed by at least one processor of a data processing system, cause the data processing system to implement the methods described herein.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### BRIEF DESCRIPTION OF THE DRAWINGS

These and other features will become more apparent from the following description in which reference is made to the appended drawings wherein:

FIG. 1A depicts an illustrative, non-limiting embodiment of a diagnostic medical device according to an aspect of the present disclosure;

FIG. 1B depicts a cross-sectional view of a support frame of the diagnostic medical device of FIG. 1A;

FIG. 2A depicts a second illustrative, non-limiting embodiment of a diagnostic medical device according to an aspect of the present device, with a support frame thereof shown in cross-section;

FIG. 2B depicts a third illustrative, non-limiting embodiment of a diagnostic medical device according to an aspect of the present device, with a support frame thereof shown in cross-section;

FIG. 2C depicts a cross-sectional side view of a support frame of the diagnostic medical device of FIG. 2A, where an appendage is received in the passage;

FIG. 3 is a flowchart showing a method of measuring properties or anatomical features of a patient's appendage;

FIG. 3A is a flowchart showing a more detailed illustrative implementation of the method of FIG. 3;

FIG. 4 depicts an illustrative, non-limiting embodiment of a wiring diagram for an illustrative, non-limiting implementation of a diagnostic device according to an aspect of the present disclosure;

FIG. 5 depicts an illustrative, non-limiting communication diagram for an illustrative, non-limiting implementation of a diagnostic device according to an aspect of the present disclosure;

FIG. 6 depicts a chart of accuracy of measured circumferences of a 28.05 cm circumference object in an illustrative, non-limiting implementation of a diagnostic device according to an aspect of the present disclosure;

FIG. 7 is an illustrative computer system in respect of which aspects of the present disclosure may be implemented; and

FIG. 8 shows an illustrative networked mobile wireless telecommunication computing device in the form of a smartphone system in respect of which aspects of the present disclosure may be implemented.

### DETAILED DESCRIPTION

Cancer survivors, such as breast cancer survivors, can live with treatment-related side effects for years after treatments have ended. For breast cancer survivors, upper extremity (UE) impairment, such as lymphedema (swelling) and decreased range of motion, is a leading cause of decreased function. A failure to recognize and manage UE impairment can lead to restrictions in activity. The device, methods, and systems described herein provide accessible, easy to use tools to help cancer survivors reduce complications and identify and manage any changes. A diagnostic medical device as described herein supports tracking and managing UE impairments in cancer survivors such as breast cancer survivors. The device is part of a system configured to measure UE function, track measurements over time, and support the provision of management strategies based on the readings obtained.

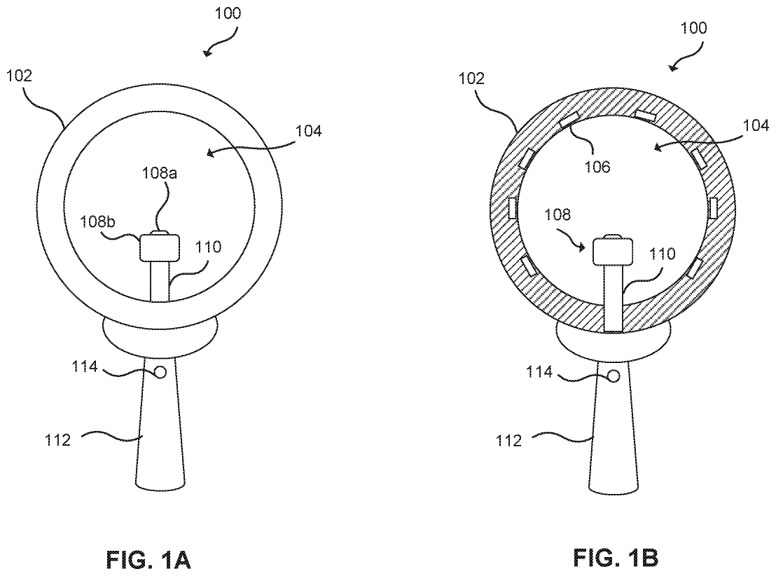

FIGS. 1A and 1B depict an illustrative, non-limiting embodiment of a diagnostic medical device 100. The diagnostic device 100 can be used by a patient or user to determine and track an impairment of the body based on at least one appendage of the user. The device may also be used by a support person or medical professional in cases where the patient or user may be unable to use the device themselves. The device is used to measure properties or anatomical features of the appendage, such as the circumference and volume of the appendage, of a human or non-human mammal. This allows a user (or a person assisting the user) to measure the size of their appendage and determine an impairment of the appendage based on the measured data and previously measured data. The terms “appendage” and “extremity” are used synonymously herein and refer to an arm (upper extremity) or leg (lower extremity) and, in the case of a non-human mammal, refer to the animal's limbs. Of note, the terms “appendage” and “extremity”, as used herein, do not encompass the head or any portion thereof, and do not encompass the penis or prepuce, or the scrotum or testes, or any portion thereof. Also of note, the device is configured to measure features of the appendage only, without measuring the trunk, head or the full body of the human or non-human mammal.

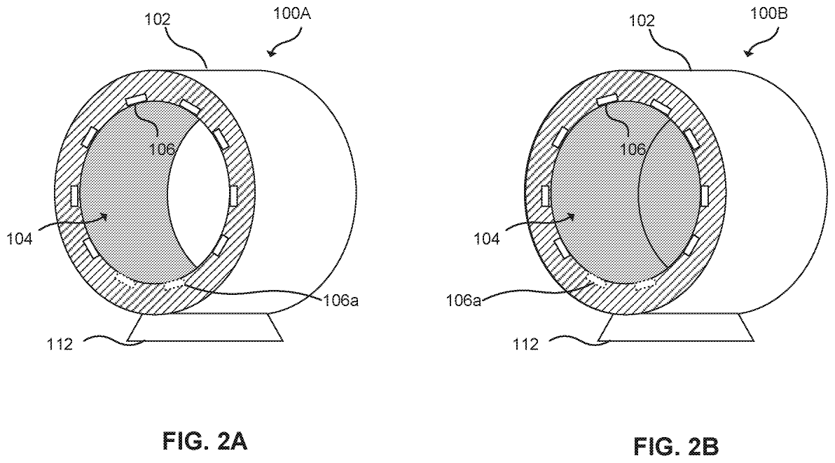

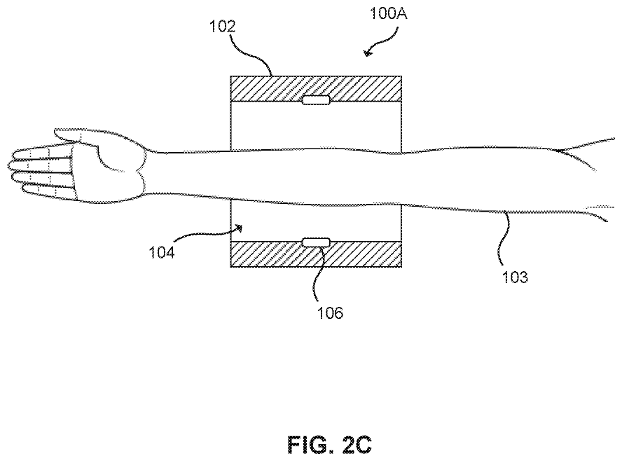

The device 100 comprises a support frame 102 which defines a passage or opening 104. The passage is sized to allow a patient's appendage to pass through and is configured to accommodate relative movement of an appendage along the passage. For example, the passage 104 may be sized and configured to accommodate circumferences across the full length of the appendage. FIGS. 2A and 2B, respectively depict further embodiments 100A, 100B of the device where the support frame 102 is a tube having both ends open (FIG. 2A) and where the support frame 102 is a tube having one open end and one closed end (FIG. 2B). FIG. 2C depicts a cross-sectional side view of the support frame 102 of the diagnostic medical device 100A of FIG. 2A, where an appendage 103 (shown as an arm but not limited thereto) is received in the passage 104. When the support frame 102 is a tube with one closed end, the length of the tube may allow for the full length of the appendage to be received within the opening 104. The full length of the appendage includes a hand as well as the measured portions of the appendage when the appendage is an arm. Although the support frame 102 is shown as a ring in FIGS. 1A and 1B and is shown as a cylindrical tube in FIGS. 2A and 2B, it will be appreciated that another shape may be used so long as the passage can accommodate the target appendage.

The support frame 102 carries a plurality of sensors 106. In one preferred embodiment, there are eight sensors although this is illustrative and not limiting. The sensors 106 are directed inwardly into the passage 104, and may, for example, be secured on or embedded in an inner surface of the support frame 102. The sensors 106 are spaced apart and are arranged to transversely surround the appendage when the appendage is received in the passage 104. The sensors 106 may be equally spaced around the perimeter of the inner surface of the support frame or may be spaced unevenly or asymmetrically around the perimeter. There may be 8 sensors spaced around the circumference of the ring as depicted in FIGS. 1A and 1B, or there may be more or fewer sensors depending on the size of the support frame 102 and the appendage to be measured. The sensors 106 are configured to cooperate together to measure a circumference of the appendage received within the passage 104. The sensors 106 may be distance sensors, such as Time of Flight (ToF) sensors, which allow for the distance between a respective sensor and an outer surface of the appendage received in the passage 104 to be measured. These distances are used to measure or calculate a circumference of the appendage at particular longitudinal positions or locations on the appendage. Data from the sensors 106 can be obtained at a plurality of longitudinal positions on the appendage to measure or calculate a circumference at each longitudinal position. The circumference measurements can be used to determine a volume of the appendage between a first longitudinal position and a last longitudinal position measured. It will be appreciated that any suitable sensors may be used to cooperate to measure the circumferences of the appendage.

The term “Time of Flight sensor”, as used herein, refers to a sensor to measure the distance between the sensor and a subject by measuring the time that elapses between emittance of a signal (e.g. light, including outside the visible spectrum, and sound) and detection of the reflected signal.

In some embodiments, the device 100 further comprises an encoder 108. In the illustrative embodiment, the encoder 108 comprises a wheel or roller 108a that rolls relative to an encoder body 108b that carries the sensor apparatus. This is merely illustrative; any suitable encoder may be used, including a magnetic encoder, an optical encoder or a capacitive encoder. The encoder 108 is carried by the support frame 102 and is configured to measure longitudinal movement of the appendage in the passage 104. The encoder 108 may be connected to a rod 110, which is secured to the device 100 such that the encoder 108 is positioned within the passage 104 as depicted in FIGS. 1A and 1B. In the illustrated embodiment, the encoder roller 108a is configured to contact a surface of the appendage and roll or move along the surface of the appendage received in the passage 104. The user may move their appendage along the passage 104 or the device 100 may be moved by the user longitudinally along the appendage to measure the circumference at a plurality of longitudinal positions along the appendage. The encoder roller 108a allows for smooth movement of the device 100 along the appendage or smooth movement of the appendage in the device 100. The encoder 108 is configured to measure the movement of the appendage in the passage. Data from the encoder represents precise information about each position measured on the appendage. The encoder 108 is carried by the support frame 102 via the rod 110 which may be a tension rod allowing the encoder wheel 108a to be in contact with the surface of the appendage throughout the length of the appendage.

In embodiments where the device does not comprise an encoder (see FIGS. 2A and 2B, for example), there may be additional sensors 106a arranged around the support frame directed inwardly to measure movement of the appendage relative to the support frame. Moreover, while FIGS. 2A and 2B show an embodiment which does not include an encoder, variations of these embodiments may be provided with an encoder.

The device 100 may further comprise a handle or mount 112 for a user to hold the device and/or to allow the user to mount the device to a surface such as a table, desk, stand or wall. The handle or mount 112 may be part of the support frame 102 or may be carried by the support frame 102. The handle or mount 112 may be permanently secured to the support frame, or may be releasably mounted to the support frame 102. When designed or shaped for a user to hold the device, the handle may be ergonomically shaped for the user.

The device 100 may further comprise one or more processors or a processing unit for receiving and processing the sensor data and/or the encoder data. The one or more processors may be housed within the support frame 102 or the handle/mount 112 and communicatively coupled to the sensors 106 and/or the encoder 108. In such embodiments, the device 100 may also be provided with a display. In some embodiments, the processor(s)/processing unit may be separate from the device 100. The device 100 may instead comprise a communication port adapted for communication with the remote processor(s)/processing unit, which may form part of a separate self-contained computing device. The sensors 106 and/or the encoder 108 may be coupled to the communication port via at least one memory adapted to store signals from the sensors and/or encoder for communication to the processor(s)/processing unit or computing device. The processor(s)/processing unit or computing device receives the signals and processes the signals to generate or calculate the circumference of the appendage at each longitudinal position measured. The processing unit or the computing device is further configured to calculate a volume of a measured portion of the appendage (between a first longitudinal position and a last longitudinal position) using the circumferences at each longitudinal position measured on the appendage.

The device 100 may further comprise an input means such as a button 114. The button 114 may be on the handle or mount 112 or may be on the support frame 102. In some embodiments, the input means is provided through a computing device in communication with the device 100, or by way of a remote or other controller separate from the device 100. The button 114 allows a user to turn on (start) or turn off (stop) the device 100. The button 114 can be used to signal to the device 100 to start measurements when the device is positioned at a first position on the appendage and to stop measurements when the device has measured a last position on the appendage. The button 114 may be further used to identify when the device is positioned at each position to signal to the device to start the measurements at the position. The device 100 may further comprise an indicator such as a light or LED (e.g. RGB LED) or a display to identify when the user can move the device 100 to a new location along the appendage. For example, while obtaining measurements at a first position, the indicator may emit a red light. When the device 100 has completed the measurement at the first position, the indicator may emit a green light, indicating that the device can be moved to a new position on the appendage. The indicator may be on the handle/mount 112 for easy access by the user, or may be on the support frame 102. The indicator may alternatively or additionally comprise a speaker configured to emit a sound to identify when the device can be moved to a new position along the appendage.

The device 100 may be sized to perform the measurements on particular extremities or appendages. As noted above, the extremities or appendages are an arm or a leg of a human or limb of non-human mammal. For example, there may be a device 100 sized for an arm, and another sized for a leg. There may be additional sizes of the device 100 depending on the size of the user. For example, there may be devices sized for larger users, smaller users, and average sized users. A smaller version of the device may have a support frame with a 168 mm inner diameter, and a larger version of the device may have a support frame with a 198 mm inner diameter. In still further embodiments, a support frame of adjustable size is contemplated, which may include additional sensors for detecting the current configuration of the support frame, or the current configuration may be manually entered into the processor(s)/processing unit or computing device for sensor calibration.

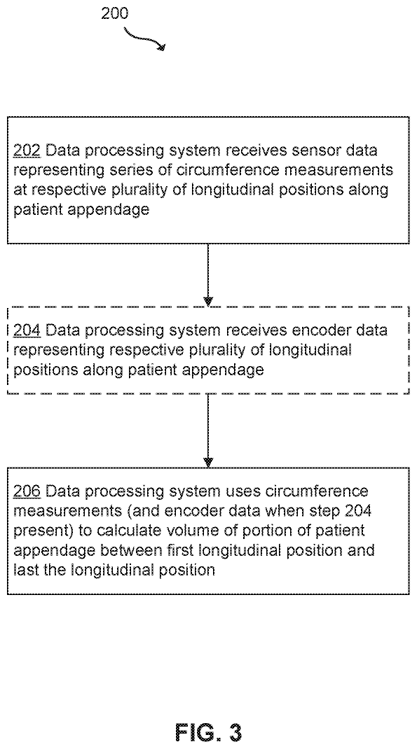

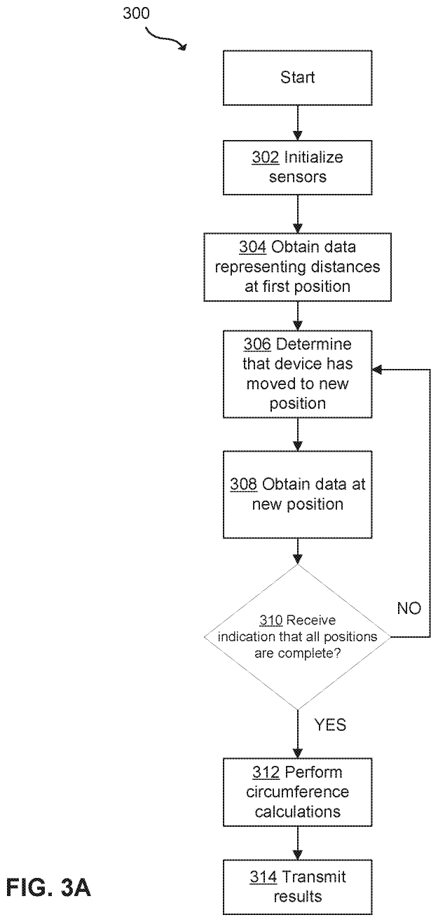

An illustrative computer-implemented method 200 of measuring properties or anatomical features of a patient's appendage is described below with reference to FIG. 3. At step 202, a data processing system receives sensor data representing a series of circumference measurements at a respective plurality of longitudinal positions along the patient appendage. The sensor data received at step 202 may comprise distance measurements from a plurality of distance sensors circumferentially surrounding the patient appendage, such as the sensors 106. At optional step 204, the data processing system receives encoder data representing the respective plurality of longitudinal positions along the patient appendage. Then, at step 206, the data processing system uses the circumference measurements and, where step 204 is present, the encoder data, to calculate a volume of a portion of the patient appendage between a first one of the longitudinal positions and a last one of the longitudinal positions. The data processing system may calculate the circumference measurements from the sensor data received at step 202, or the sensor data received at step 202 may comprise precalculated circumference measurements. Where step 204 is present, steps 202 and 204 may be performed in reverse order, or substantially simultaneously.

Thus, where step 204 is present, the method 200 comprises receiving, at a data processing system, sensor data (step 202) and encoder data (step 204). The sensor data represents a series of circumference measurements at a respective plurality of longitudinal positions along the patient's appendage. The circumference measurements are obtained at discrete positions along the appendage or may be obtained via a scan of the appendage. The encoder data represents the respective plurality of longitudinal positions along the patient appendage. The sensor data may comprise distance measurements from a plurality of sensors that are circumferentially arranged around the appendage. The data processing system can use the sensor data to calculate the circumference measurements at the respective longitudinal positions. In some embodiments, the sensor data comprises precalculated circumference measurements of the appendage.

The encoder data in conjunction with the circumference measurements is used by the data processing system to calculate the volume of a portion of the patient appendage between a first one of the longitudinal positions and a last one of the longitudinal positions. The above methods can be repeated over a period of time to obtain circumference measurements and volumes of the appendage over the period of time. The data from particular times can be compared to data from other times to determine an impairment of the patient, or to track any known impairment of the patient, and/or to provide information in support of treatment of the impairment of the patient. The methods and comparisons can be performed in real-time for efficient and accurate determinations, tracking, and supporting treatments of impairments such as lymphedema and range of motion issues.

FIG. 3A is a more detailed flowchart depicting an illustrative, non-limiting implementation, denoted at reference 300, of the method 200 shown in FIG. 3 for measuring properties or anatomical features of a patient's appendage using the device described herein. The method 300 may be implemented using the devices 100, 100A, 100B shown in FIGS. 1, 2A, and 2B, for example. The properties or anatomical features may be circumferences and a volume of the appendage being measured. The method starts when the device 100, 100A, 100B has been positioned at a first location or position on a user's appendage (e.g. appendage 103 in FIG. 2C). The user (or a person assisting the user) may indicate that the device is at the first position using the input means 114 or the device 100, 100A, 100B may automatically determine that the device is at the first position when the device is still and the sensors detect an appendage 103 and/or the encoder 108 (where present) is in contact with a surface of the appendage.

At step 302, the sensors 106 are initialized to cooperate to obtain data representing a volume of a portion of the patient appendage 103 by measuring a plurality of circumferences of the patient appendage. At step 304, each sensor 106 obtains sensor data representing a distance between the respective sensor and a surface of the appendage 103. In some embodiments, the sensors 106 may initialize before the device 100 is at a first position on the appendage 103. The user may use the input means 114 to initialize the sensors before the device 100, 100A, 100B is positioned around the appendage 103. For example, in the case of an arm of a breast cancer survivor, the first position may be at the wrist crease of the arm 103, and the device 100, 100A, 100B may move along the arm 103 (or the arm 103 may move along the device) ending at the axilla/upper shoulder, or vice versa.

While the sensors 106 are obtaining the data at the first position, the indicator may indicate that the device is in use and cannot be moved. Once each sensor 106 has completed the measurement, the indicator may indicate that the measurements are complete for the first position, and the user is to move the device 100, 100A, 100B or their appendage 103 to a second position a distance away from the first position on the appendage 103.

After the user has moved the device 100, 100A, 100B or their appendage 103 to the next position, at step 306 it may be determined (e.g. from the encoder) that the device 100, 100A, 100B is positioned at the next position and the device 100, 100A, 100B may obtain precise information about the position along the appendage 103 such as the distance between the previous position and the new position. Step 308 is similar to step 304, as the sensors 106 are configured to obtain sensor data representing distances between the respective sensor 106 and a surface of the appendage 103 at the second position. Steps 306 and 308 are repeated for a portion of the appendage 103. The portion of the appendage 103 is between the first position and a last position of the appendage 103. The first position may be a wrist crease or upper shoulder of an arm, and the last position may be anywhere between the first position and the respective upper shoulder or wrist crease of the arm. This is merely illustrative and not limiting. There may be a predetermined number of positions for measurement based on the portion of the appendage 103 being measured. The user may be trained to determine the number of positions before using the device 100, 100A, 100B, or the device 100, 100A, 100B may be configured to determine and indicate the number of positions based on an input of the appendage being measured.

With every specified distance the device 100, 100A, 100B has moved along the appendage 103 (or vice versa), the sensors 106 are configured to obtain sensor data. Once the sensor data has been obtained for all positions along the appendage 103, at step 310 an indication (e.g. via the input means 114) that all the positions are completed can be received. In some embodiments, the device 100 may be configured to automatically determine that all positions have been completed (e.g. based on the total distance the encoder has travelled, the number of positions that have been completed or other suitable indication).

For each position, the one or more processors receive the data from the sensors 106 (and the data from the encoder where present). The data may be transmitted during or after each position is complete, or after all the positions have been completed. It will be appreciated that obtaining the data at all positions may take a short amount of time such as 10-15 seconds; longer durations are also contemplated although less preferred. The short amount of time allows for a user to easily analyze their appendage 103. The sensor data allows for the circumferences of the appendage 103 to be measured and calculated. The circumference at each position on the appendage 103 may be estimated using an arc length formula. Using the sensors, a radius at each location can be found. Since the angle between the sensors and/or the location of each sensor on the support frame is known, this information can be used to find an arc length of the appendage. For example, in one embodiment, circumference measurements can be taken every 1 cm; this is merely illustrative and not limiting. The appendage, such as an arm, may be modelled as frustums for segmental extremity volume calculation. Calculation of volume from the circumference measurements is within the capability of one of ordinary skill in the art, now informed by the present disclosure.

Using the data from the sensors 106 (and the encoder 108 when present), at step 312 the circumference at each longitudinal position along the appendage is measured or calculated. The calculation is performed by the one or more processors, and the results can be transmitted to a local display, a server, PC, and/or other computing device (314) for storage and further analysis. The one or more processors can further calculate a volume of the appendage using at least the circumference measurements for each of the plurality of longitudinal positions. In some embodiments, the server, PC or computing device may instead calculate the volume of the appendage using at least the received circumference measurement for each of the plurality of longitudinal positions.

The method described herein may be repeated at different times to analyze any differences in the circumferences and volume of the user's appendage. The data is used to determine the presence and degree of extremity impairment of a user. The method may be performed once a day, week, month, or other based on the extremity, the user's symptoms, and/or recommendations by a medical professional.

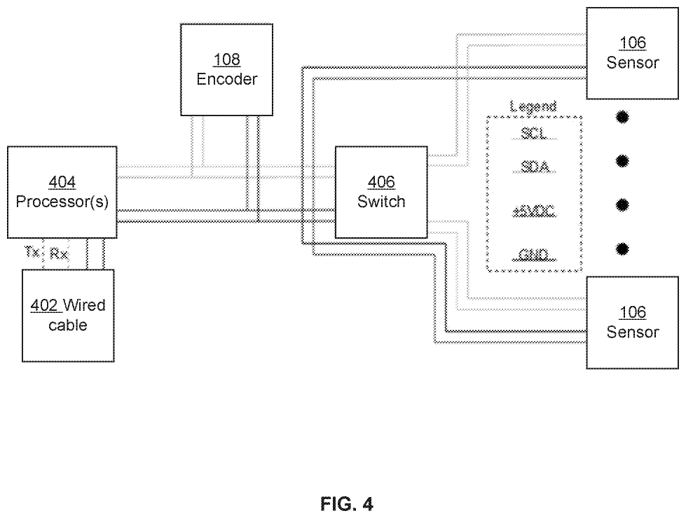

FIG. 4 depicts an exemplary embodiment of a wiring diagram for an illustrative, non-limiting implementation of the device 100 described herein. The communications of the device may be provided wirelessly for a network (e.g. Bluetooth® wireless module) or wired through, for example, a USB-C or other similar cable 402. The device 100 is configured to communicate with a computing device to transmit data and information obtained and calculated by the device. The power may be supplied to the device by a battery or other power source within the device (e.g. the handle or mount), or may be provided through a wired cable. The power supplied may be 5V. The device may comprise one or more processors 404 as described above. The sensors 106 of the device may communicate with the one or more processors 404 through a switch 406 such as an inter-integrated circuit (I²C) switch using two bus lines (serial clock or “SCL” and serial data or “SDA”). The sensors 106 may be connected to the switch 406 which connects to the one or more processors 404. The sensors 106 may, for, example, be a STMicroelectronics VL6180X sensor, offered by STMicroelectronics, having an address at Chemin du Champ-des-Filles 39, 1228 Plan-les-Ouates, Switzerland. The STMicroelectronics VL6180X sensor is offered for sale at https://estore.st.com/en/. The STMicroelectronics VL6180X sensor is merely an example and is not limiting, other suitable processors, microprocessors and microcontrollers are contemplated. The encoder 108 of the device 100 may be in direct communication with the one or more processors 404, bypassing the I²C switch as shown in FIG. 4, or indirectly communicatively coupled to the one or more processors 404. In some embodiments, the device 100 comprises a communications port coupled to the sensors and encoder (where present) to transmit data to a communications port of a computing device having one or more processors. The communications port may be a wired communication port, such as a cable port, or a wireless communication port, such as a wireless radio. The sensors and encoder may be coupled to the communication port through at least one memory. The memory may temporarily store signals from the sensors and encoder before transmitting any data to the computing device. In one preferred embodiment, the one or more processors 404 may be provided by an Arduino® Nano ESP32®, which includes memory, a USB-C communication port, as well as a u-blox® NORA-W106 module and provides both Bluetooth® & Wi-Fi® connectivity, in addition to embedding an antenna. The Arduino Nano ESP32 is offered for sale at https://store-usa.arduino.cc. The Arduino Nano ESP32 is merely an example and is not limiting.

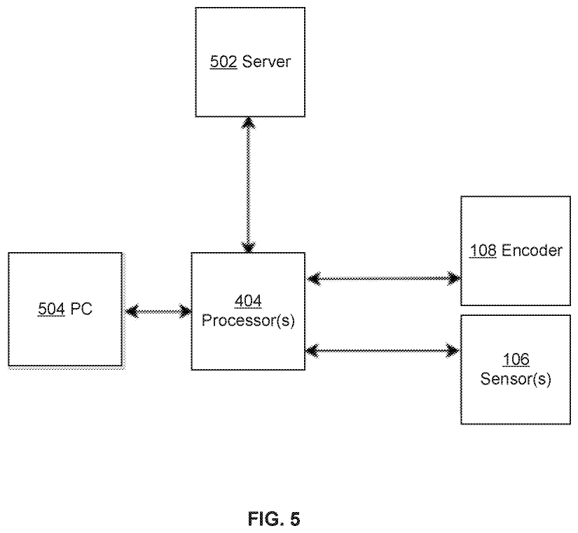

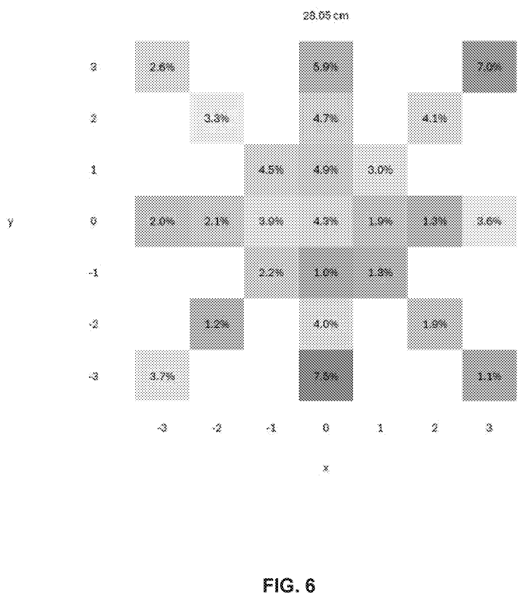

FIG. 5 depicts an illustrative, non-limiting communication diagram for an illustrative, non-limiting implementation of the device 100 described herein. There may be multiple different communication protocols used between the elements of the device. For example, an I²C communication protocol may be used to connect the sensors 106 to the same bus. The communication for the sensors decreases the wiring in the system and allows for easier access to individual sensors. HTTP (Hypertext Transfer Protocol) may be used to wirelessly create and update a server or webserver 502 allowing the user to receive measurement feedback. UART (Universal asynchronous receiver-transmitter) may be used for debugging purposes when connected to a personal computer (PC) or other computing device 504. A communication protocol for debugging allows for more data to be shown to the user to better intercept fault measurements. Although three communication protocols are identified above, it will be appreciated that any number and any suitable communication protocol may instead be used.

The device is configured to communicate with a computing device (such as a server, desktop or laptop computer or smartphone) having a health application or software. The sensor and encoder data may be transmitted to the server for analysis of the measurements obtained such that feedback can be provided to the user about the measurements. The computing device may be configured to provide real-time updates on the obtained and/or processed data. For example, if a measurement appears obscure or does not correspond to other measurements taken, the user may receive an indication to redo one or more measurements with the device. The determined data may also be transmitted to a computing device for review and analysis by the user and/or medical professional. The data collected and processed over various measurement sessions by the device and user are used to track changes in the measurements to monitor progression of any extremity impairment. The health application or software may allow the user or a clinician to view the changes in the volume and circumference of the extremity to determine the presence of a potential impairment of the extremity, and the degree of extremity impairment. In some embodiments, the system is configured to automatically determine the presence of an impairment, and the degree of extremity impairment based on the circumference measurements and volume. The user may view the data of their extremity through a user interface of the application on the computing device.

The device may be supplied to users with instructions on the system features. A medical professional, such as a registered physiotherapist trained on the system including the device, can provide a user with information on how to use the system, show the user how to perform movements with proper form and educate on signs/symptoms of various issues such as lymphedema. The system allows users to log in, measure range of motion/circumference, access stored data, and more to identify and prevent and treat any range of motion issues. The health application or software may further use questionnaires to better analyze and store information about the user, and better understand the usability of the system.

The device is part of the system that allows a user to measure UE or other function (range of motion and volume) independently. The reliability of the system was tested with in-person visits to manually measure the outcomes to determine reliability and validity of the system. Manual measurements were completed on the same days as system measurements.

FIG. 6 depicts a chart of an accuracy of measured circumferences of a 28.05 cm circumference object in an exemplary embodiment of the disclosure. Controlled measurements produce a repeatable circumference calculation. During testing, 0.097 cm deviation at center was measured, as was 0.083 cm deviation 1 cm from center, 0.174 cm deviation 2 cm from center, and 0.209 cm deviation 3 cm from center in repeated trials (center referring to the center of a ring-shaped support frame).

Aspects of the present technology may be embodied within a device, a system, a method, a computer program product or any combination thereof. The computer program product may include a computer readable storage medium or media having computer readable program instructions thereon for causing a processor to carry out aspects of the present technology. The computer readable storage medium can be a tangible, non-transitory device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present technology may be assembler instructions, instruction-set-architecture (ISA) instructions, machine instructions, machine dependent instructions, microcode, firmware instructions, state-setting data, or either source code or object code written in any combination of one or more programming languages, including an object oriented programming language or a conventional procedural programming language. The computer readable program instructions may execute entirely on the user's computer or device, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to implement aspects of the present technology.

Aspects of the present technology are described herein with reference to flowchart illustrations and/or block diagrams of methods, apparatus (systems) and computer program products according to various embodiments. In this regard, the flowchart and block diagrams in the Figures illustrate architecture, functionality, and operation of possible implementations of systems, methods and computer program products according to various embodiments of the present technology. For instance, each block in the flowchart or block diagrams may represent a module, segment, or portion of instructions, which comprises one or more executable instructions for implementing the specified logical function(s). It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Some specific examples of the foregoing may have been noted above but any such noted examples are not necessarily the only such examples. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It also will be understood that each block of the flowchart illustrations and/or block diagrams, and combinations of blocks in the flowchart illustrations and/or block diagrams, can be implemented by computer program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, other programmable data processing apparatus, or other devices to function in a particular manner, such that the instructions stored in the computer readable storage medium produce an article of manufacture including instructions which implement aspects of the functions/acts specified in the flowchart and/or block diagram block or blocks. The computer readable program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

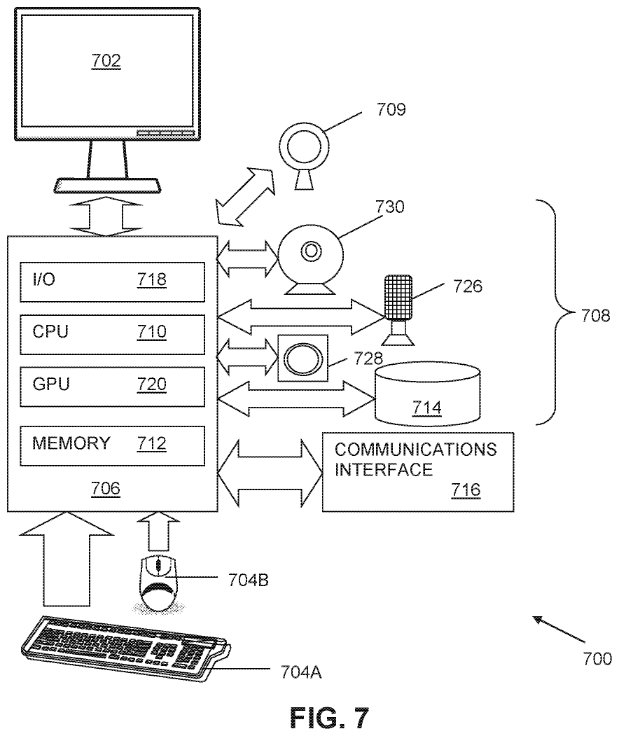

An illustrative computer system in respect of which the technology herein described may be implemented is presented as a block diagram in FIG. 7. The illustrative computer system is denoted generally by reference numeral 700 and includes a display 702, input devices in the form of keyboard 704A and pointing device 704B, computer 706, external devices 708, and a diagnostic medical device 709 as described herein. While pointing device 704B is depicted as a mouse, it will be appreciated that other types of pointing device, or a touch screen, may also be used.

The computer 706 may contain one or more processors or microprocessors, such as a central processing unit (CPU) 710. The CPU 710 performs arithmetic calculations and control functions to execute software stored in an internal memory 712, preferably random access memory (RAM) and/or read only memory (ROM), and possibly additional memory 714. The additional memory 714 may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This additional memory 714 may be physically internal to the computer 706, or external as shown in FIG. 7, or both.

The computer system 700 may also include other similar means for allowing computer programs or other instructions to be loaded. Such means can include, for example, a communications interface 716 which allows software and data to be transferred between the computer system 700 and external systems and networks. Examples of communications interface 716 can include a modem, a network interface such as an Ethernet card, a wireless communication interface, or a serial or parallel communications port. Software and data transferred via communications interface 716 are in the form of signals which can be electronic, acoustic, electromagnetic, optical or other signals capable of being received by communications interface 716. Multiple interfaces, of course, can be provided on a single computer system 700.

Input and output to and from the computer 706 is administered by the input/output (I/O) inter-face 718. This I/O interface 718 administers control of the display 702, keyboard 704A, external devices 708 and other such components of the computer system 700. The computer 706 also includes a graphical processing unit (GPU) 720. The latter may also be used for computational purposes as an adjunct to, or instead of, the (CPU) 710, for mathematical calculations.

The external devices 708 include a microphone 726, a speaker 728 and a camera 730. Although shown as external devices, they may alternatively be built in as part of the hardware of the computer system 700. The computer system further includes the diagnostic medical device 709. The device 709 is configured to obtain and in some embodiments, process data about an appendage of a patient. The device 709 may be wirelessly coupled or directly coupled (wired, e.g. USB-C) to the computer 706 to transmit and receive data and instructions. The device 709 may have its own communications port for communicating with the computer 706. The device 709 may comprise a processing unit for processing the data before transmitting any data to the computer 706.

It will be appreciated that the various components of the computer system 700 are coupled to one another either directly or by coupling to suitable buses.

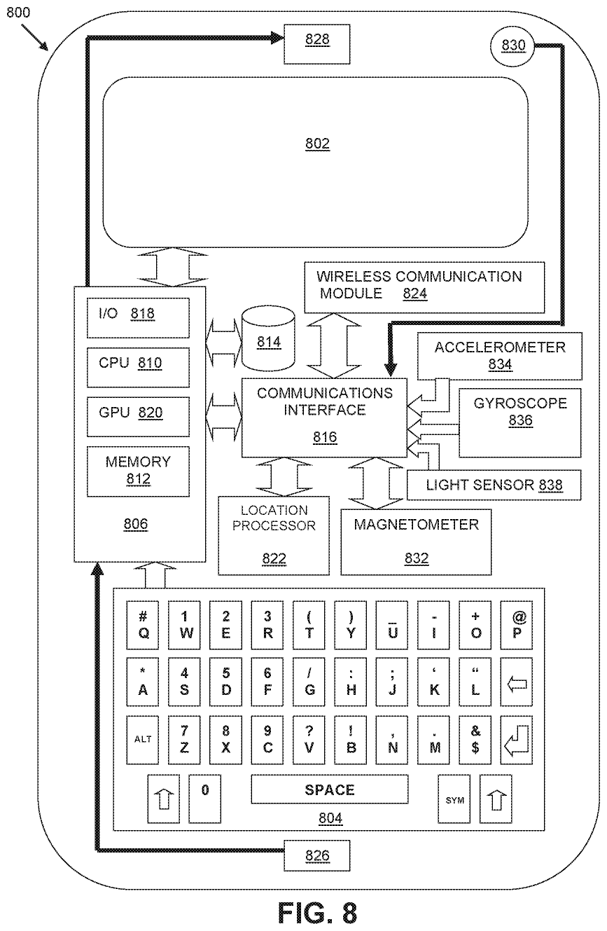

FIG. 8 shows an illustrative networked mobile wireless telecommunication computing device in the form of a smartphone 800. The smartphone 800 includes a display 802, an input device in the form of keyboard 804 and an onboard computer system 806. The display 802 may be a touchscreen display and thereby serve as an additional input device, or as an alternative to the keyboard 804. The onboard computer system 806 comprises a central processing unit (CPU) 810 having one or more processors or microprocessors for performing arithmetic calculations and control functions to execute software stored in an internal memory 812, preferably random access memory (RAM) and/or read only memory (ROM) is coupled to additional memory 814 which will typically comprise flash memory, which may be integrated into the smartphone 800 or may comprise a removable flash card, or both. The smartphone 800 also includes a communications interface 816 which allows software and data to be transferred between the smartphone 800 and external systems and networks (such as a diagnostic medical device as described herein). The communications interface 816 is coupled to one or more wireless communication modules 824, which will typically comprise a wireless radio for connecting to one or more of a cellular network, a wireless digital network or a Wi-Fi network. A device (e.g. the device 100) as described herein may communicate with the smartphone 800 via the wireless communication module(s), for example. The communications interface 816 will also typically enable a wired connection of the smartphone 800 to an external computer system. A microphone 826 and speaker 828 are coupled to the onboard computer system 806 to support the telephone functions managed by the onboard computer system 806, and a location processor 822 (e.g. including GPS receiver hardware) may also be coupled to the communications interface 816 to support navigation operations by the onboard computer system 806. One or more cameras 830 (e.g. front-facing and/or rear facing cameras) may also be coupled to the onboard computer system 806, as may be one or more of a magnetometer 832, accelerometer 834, gyroscope 836 and light sensor 838. Input and output to and from the onboard computer system 806 is administered by the input/output (I/O) interface 818, which administers control of the display 802, keyboard 804, microphone 826, speaker 828, camera 830, magnetometer 832, accelerometer 834, gyroscope 836 and light sensor 838. The onboard computer system 806 may also include a separate graphical processing unit (GPU) 820. The various components are coupled to one another either directly or by coupling to suitable buses.

The term “computer system”, “data processing system” and related terms, as used herein, is not limited to any particular type of computer system and encompasses servers, desktop computers, laptop computers, networked mobile wireless telecommunication computing devices such as smartphones, tablet computers, as well as other types of computer systems.

Thus, computer readable program code for implementing aspects of the technology described herein may be contained or stored in the memory 812 of the onboard computer system 806 of the smartphone 800 or the memory 712 of the computer 706, or on a computer usable or computer readable medium external to the onboard computer system 806 of the smartphone 800 or the computer 706, or on any combination thereof.

Finally, the terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description has been presented for purposes of illustration and description, but is not intended to be exhaustive or limited to the form disclosed. Many modifications and variations will be apparent to those of ordinary skill in the art without departing from the scope of the claims. The embodiment was chosen and described in order to best explain the principles of the technology and the practical application, and to enable others of ordinary skill in the art to understand the technology for various embodiments with various modifications as are suited to the particular use contemplated.

One or more currently preferred embodiments have been described by way of example. It will be apparent to persons skilled in the art that a number of variations and modifications can be made without departing from the scope of the claims. In construing the claims, it is to be understood that the use of a computer to implement the embodiments described herein is essential.

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

It will be understood that any component defined herein as being included may be explicitly excluded by way of proviso or negative limitation, such as any specific compounds or method steps, whether implicitly or explicitly defined herein.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### CITATIONS

The following list of references is provided for convenience only, and without admission that any of the references constitutes prior art or is relevant to the invention as claimed.
1. Canadian Cancer Society. Breast cancer statistics [Internet]. 2022. Available from: https://cancer.ca/en/cancer-information/cancer-types/breast/statistics.
2. Cella D, Fallowfield LJ. Recognition and management of treatment-related side effects for breast cancer patients receiving adjuvant endocrine therapy. Breast Cancer Res Treat. 2008;107(2):167-180.
3. Ewertz M, Jensen AB. Late effects of breast cancer treatment and potentials for rehabilitation. Acta Oncol. 2011;50(2):187-193.
4. McNeely M, Campbell K, Ospina M, Rowe B, Dabbs K, Klassen T, et al. Exercise interventions for upper-limb dysfunction due to breast cancer treatment. Cochrane Syst Rev. 2010;16(6):CD005211.
5. Reitman JS, Dijkstra PU, Hoekstra HJ, Wisma WH, Szabo BG, Groothoff JW, et al. Late morbidity after treatment of breast cancer in relation to daily activities and quality of life: a systematic review. Eur J Surg Oncol. 2003;29:229-38.
6. DiSipio T, Rye S, Newman B, Hayes S. Incidence of unilateral arm lyphoedema after breast cancer: A systematic review and meta-analysis. Lancet Oncol. 2013;14(6):500-515.
7. Lee TS, Kilbreath SL, Refshauge KM, Herbert RD, Beith JM. Prognosis of the upper limb following surgery and radiation for breast cancer. Breast Cancer Res Treat. 2008;110(1):19-37.
8. Pereira A, Koifman R, Bergmann A. Incidence and risk factors of lymphedema after breast cancer treatment: 10 years of follow-up. Breast. 2017;36:67-73.
9. Smoot B, Paul S, Aouizerat B, Dunn L, Elboim C, Schmidt B, et al. Predictors of altered upper extremity function during the first year after breast cancer treatment. Am J Phys Med Rehabil. 2016;95(9):639-655.
10. Cheville AL, Beck LA, Petersen TL, Marks RS, Gamble GL. The detection and treatment of cancer-related functional problems in an outpatient setting. Support Care Cancer. 2009;17:61-67.
11. Soran A, Ozmen T, McGuire KP, Diego EJ, McAuliffe PF, Bonaventura M, et al. The importance of detection of subclinical lymphedema for the prevention of breast cancer-related clinical lymphedema after axillary lymph node dissection; a prospective observational study. Lymphat Res Biol. 2014;12:289-294.
12. Klasnja P, Pratt W. Healthcare in the pocket: mapping the space of mobile-phone health interventions. J Biomed Inform. 2012;45(1):184-98.
13. Fiordelli M, Diviani N, Schulz P. Mapping mHealth research: a decade of evolution. J Med Internet Res. 2013;15(5).
14. Marcolino M, Oliveira J, D'Agostino M, Ribeiro AL, Alkmim M, Novillo-Ortiz D. The impact of mHealth interventions: systematic review of systematic reviews. JMIR Mhealth Uhealth. 2018;6(1):e23.
15. Gerber LH, Stout NL, Schmitz KH, Stricker CT. Integrating a prospective surveillance model for rehabilitation into breast cancer survivorship care. Cancer. 2012;118:2201-2206
16. Canadian Cancer Society's Advisory Committee on Cancer Statistics. Canadian Cancer Statistics 2015 [Internet]. June 2015. Available from: https://www.cancer.ca/~/media/cancer.ca/CW/cancer%20information/cancer%20101/Canadian%20cancer%20statistics/Canadian-Cancer-Statistics-2015-EN.pdf?la=en
17. Mori T, Lustman A, Katz-Leurer M. Self-measurement of upper extremity volume in women post-breast cancer: reliability and validity study. Physiother Theory Pract. 2015;31:283-287.
18. Bolette SR, McNeely M, Camp P, Midtgaard J, Campbell K. Self-measured arm circumference in women with breast cancer is reliable and valid. Phys Ther. 2019;99:240-253.
19. Hardcastle SJ, Jiménez-Castuera R, Maxwell-Smith C, Bulsara MK, Hince D. Fitbit wear-time and patterns of activity in cancer survivors throughout a physical activity intervention and follow-up: Exploratory analysis from a randomised controlled trial. PloS ONE. 2020;15(10):e0240967.
20. App Store Preview. Tape Measure [Internet]. 2020. Available at: https://apps.apple.com/us/app/tape-measure/id1271546805
21. App Store Preview. EasyMeasure-Camera Ruler [Internet]. 2020. Available at: https://apps.apple.com/us/app/easymeasure-camera-ruler/id349530105
22. App Store Preview. Goniometer Plus [Internet]. 2020. Available at: https://apps.apple.com/us/app/goniometer-plus/id1081939665
23. Ishii K, Oka H, Honda Y, et al. Accuracy and reliability of a smartphone application for measuring knee joint angle. J Phys Ther Scie. 2021;33(5).
24. Willmon RH, Gulick DR, Paterson ML, et al. Validity and reliability of 2 goniometric mobile apps: Device, application, and examiner factors. J Sport Rehabil. 2016;25(4):371-379.
25. Vohralik SL, Bowen AR, Burns J, et al. Reliability and validity of a smartphone app to measure joint range. Am J Phys Med Rehabil. 2015;94(4):325-330.
26. Reid S, Egan B. The validity and reliability of DrGoniometer, a smartphone application, for measuring forearm supination. J Hand Therap. 2019;32(1):110-117.

What is claimed is:

1. A diagnostic medical device, comprising:
a support frame, the support frame defining a passage therein, wherein the passage is configured to accommodate relative movement of a patient appendage along the passage; and
a plurality of spaced-apart sensors carried by the support frame, the plurality of sensors directed inwardly into the passage and arranged to transversely surround the patient appendage when received in the passage, wherein the sensors are configured to cooperate to obtain data representing a volume of a measured portion of the patient appendage by measuring a plurality of circumferences of the patient appendage at a respective plurality of longitudinal positions of the patient appendage relative to the passage when the patient appendage is received in the passage.

2. The device of claim 1, further comprising an encoder carried by the support frame and configured to measure longitudinal movement of the patient appendage.

3. The device of claim 2, wherein the encoder comprises a roller for contacting and rolling along a surface of the patient appendage.

4. The device of claim 1, further comprising at least one processing unit coupled to the plurality of sensors and configured to receive signals from the plurality of sensors, and to process the signals to generate the circumference of the patient appendage at each of the plurality of longitudinal positions.

5. The device of claim 4, wherein the at least one processing unit is further configured to calculate the volume of the measured portion of the patient appendage using the circumferences of the patient appendage at each of the longitudinal positions.

6. The device of claim 1, wherein the plurality of sensors is coupled to a communication port adapted for communication to a computing device.

7. The device of claim 6, wherein the plurality of sensors is coupled to the communications port through at least one memory adapted to store signals from the sensors for communication to the computing device.

8. The device of claim 2, wherein the plurality of sensors and the encoder are coupled to a communication port adapted for communication to a computing device.

9. The device of claim 8, wherein the plurality of sensors and the encoder are coupled to the communications port through at least one memory adapted to store signals from the sensors for communication to the computing device.

10. The device of claim 1, wherein the support frame comprises a ring.

11. The device of claim 1, wherein the support frame comprises a tube having an open first end and a closed second end.

12. The device of claim 1, wherein the support frame comprises a tube having an open first end and an open second end.

13. The device of claim 1, wherein the patient appendage is an arm, and a first longitudinal position of the plurality of longitudinal positions is at a wrist crease or an upper shoulder of the arm.

14. The device of claim 1, wherein the plurality of sensors are distance sensors.

15. The device of claim 14, wherein the plurality of sensors are Time of Flight sensors.

16. A method for measuring anatomical features of a patient appendage, the method comprising:
receiving, at a data processing system, sensor data representing a series of circumference measurements at a respective plurality of longitudinal positions along the patient appendage; and
the data processing system using the circumference measurements to calculate a volume of a portion of the patient appendage between a first one of the longitudinal positions and a last one of the longitudinal positions.

17. The method of claim 16, further comprising:
receiving, at the data processing system, encoder data representing the respective plurality of longitudinal positions along the patient appendage, wherein
the data processing system uses the encoder data in conjunction with the circumference measurements to calculate the volume of the portion of the patient appendage between the first one of the longitudinal positions and the last one of the longitudinal positions.

18. The method of claim 16, wherein the sensor data comprises distance measurements from a plurality of distance sensors circumferentially surrounding the patient appendage.

19. The method of claim 16, wherein the data processing system calculates the circumference measurements from the sensor data.

20. The method of claim 16, wherein the sensor data comprises precalculated circumference measurements.

21. A computer readable memory storing computer executable instructions thereon that when executed by a computer cause the computer to perform the method of claim 16.

22. A data processing system comprising at least one processor and memory coupled to the at least one processor, wherein the memory contains instructions which, when executed by the at least one processor, cause the data processing system to implement the method of claim 16.

23. At least one tangible, non-transitory computer-readable medium embodying instructions which, when executed by at least one processor of a data processing system, cause the data processing system to implement the method of claim 16.
